(19) United States
(12) Patent Application Publication
(10) Pub. No.: US 2022/0241168 A1
(43) Pub. Date: Aug. 4, 2022

(54) COMPOSITION FOR STIMULATING BETA-ENDORPHIN PRODUCTION COMPRISING SANSHOOL

(71) Applicant: AMOREPACIFIC CORPORATION, Seoul (KR)

(72) Inventors: Hyung Su KIM, Yongin-si (KR); Si Young CHO, Yongin-si (KR); Young Gyu KANG, Yongin-si (KR); Hyuk KIM, Yongin-si (KR); Euidong SON, Yongin-si (KR)

(73) Assignee: AMOREPACIFIC CORPORATION, Seoul (KR)

(21) Appl. No.: 17/589,202

(22) Filed: Jan. 31, 2022

(30) Foreign Application Priority Data
Feb. 4, 2021 (KR) 10-2021-0015881

(51) Int. Cl. A61K 8/42; A61Q 7/00; A61Q 19/04; A61Q 19/00; A23L 33/00; A23L 33/10

(52) U.S. Cl. CPC A61K 8/42; A61Q 7/00; A61Q 19/04; A23V 2002/00; A23L 33/40; A23L 33/10; A61K 2800/92; A61Q 19/005

(57) ABSTRACT

A composition includes, as an active ingredient, a substance capable of providing a useful effect to the skin by stimulating production of beta (β)-endorphin. The composition containing, as an active ingredient, sanshool, an isomer thereof, a hydrate thereof, or a solvate thereof stimulates production of β-endorphin when applied into the body, thereby providing useful effects to the skin, such as relieving skin irritation from the outside and providing a skin soothing effect.

### CROSS REFERENCE TO RELATED APPLICATION

This application claims priority to Korean Patent Application No. 10-2021-0015881, filed on Feb. 4, 2021, the entire content of which is incorporated herein by reference.

### BACKGROUND

### Field

The present disclosure relates to a composition for stimulating production of beta-endorphin and a method for stimulating production of beta-endorphin in a subject.

### Description of the Related Art

The skin is the largest organ of the body, and substances released through the skin have a great effect on the body. Beta (β)-endorphin, which is an endogenous opioid neuropeptide produced by certain neurons in the central nervous system and the peripheral nervous system, is known to relieve inflammation and pain in the body and to protect the skin barrier from external stress.

### SUMMARY

In one aspect, an object to be achieved by the present disclosure is to provide a composition comprising, as an active ingredient, a substance capable of providing a useful effect to the skin by stimulating production of beta-endorphin through skin application.

An embodiment of the present disclosure provides a composition for stimulating production of beta-endorphin comprising, as an active ingredient, sanshool, an isomer thereof, a hydrate thereof, or a solvate thereof.

An embodiment of the present disclosure comprises, as an active ingredient, sanshool, an isomer thereof, a hydrate thereof, or a solvate thereof, thereby stimulating production of beta-endorphin and providing a useful effect to the skin.

### BRIEF DESCRIPTION OF THE DRAWINGS

FIG. 1 is a diagram showing results obtained by measuring efficacy of stimulating production of beta (β)-endorphin of cannabidiol (CBD) (Comparative Example 1), honokiol (Comparative Example 2), magnolol (Comparative Example 3), palmitoylethanolamide (PEA) (Comparative Example 4), which are known as cannabinoid receptor type 2 (CB2) agonists, as comparative examples of the present disclosure.

FIG. 2 is a diagram showing results obtained by measuring efficacy of stimulating production of β-endorphin of hydroxy-alpha (α)-sanshool (Example 1), hydroxy-gamma (γ)-sanshool (Example 2), hydroxy-β-sanshool (Example 3), and hydroxy-epsilon (E)-sanshool (Example 4) as an embodiment of the present disclosure.

FIG. 3 is a diagram showing a comparison of efficacy of stimulating of production of β-endorphin according to content of hydroxy-α-sanshool as an embodiment of the present disclosure.

### DETAILED DESCRIPTION

Hereinafter, embodiments of the present disclosure are described in detail.

Embodiments of the present disclosure, which are disclosed in the present specification, are illustrated for the purpose of explanation only, and the embodiments of the present disclosure may be embodied in various embodiments and should not be construed as being limited to the embodiments described herein. The present disclosure allows for various changes and numerous embodiments, but the embodiments are not intended to limit the present disclosure to particular modes of practice, and are to be appreciated that all modifications, equivalents, and substitutes that do not depart from the spirit and technical scope of the present disclosure are encompassed. An expression used in the singular encompasses the expression of the plural, unless it has a clearly different meaning in the context. In the present specification, it is to be understood that the terms such as “including,” “comprising,” and “having” are intended to indicate the existence of the features, materials, numbers, steps, elements, or combinations thereof disclosed in the specification, and are not intended to preclude the possibility that one or more other features, materials, numbers, steps, operations, elements, or combinations thereof may exist or may be added.

An embodiment of the present disclosure provides a composition for stimulating production of beta (β)-endorphin comprising, as an active ingredient, sanshool, an isomer thereof, a hydrate thereof, or a solvate thereof.

In the present specification, the term “skin” refers to a tissue covering the body surface of an animal, and is the broadest concept encompassing all tissues covering the body surface, such as the face, scalp, or body.

In the present specification, the term “isomer” particularly comprises, in addition to optical isomers (e.g., essentially pure enantiomers, essentially pure diastereomers, or a mixture thereof), conformation isomers (i.e., isomers that differ only in the angle of one or more chemical bonds), position isomers (particularly, tautomers), or geometric isomers (e.g., cis-trans isomers).

In the present specification, when used in reference to, for example, enantiomers or diastereomers, the term “essentially pure” means that a specific compound, for example, enantiomers or diastereomers, is present by about 90% or more, preferably, about 95% or more, more preferably, about 97% or more or about 98% or more, more preferably, about 99% or more, more preferably, about 99.5% or more (w/w).

In the present specification, the term “hydrate” refers to a compound to which water is bound, and is a broad concept encompassing inclusion compounds that do not have a chemical bond between water and the compound.

In the present specification, the term “solvate” refers to a higher-order compound formed between a molecule or ion of a solute and a molecule or ion of a solvent.

Another embodiment may provide the use of the sanshool, the isomer thereof, the hydrate thereof, or the solvate thereof for use in preparation of a composition for stimulating production of β-endorphin. Another embodiment may provide a method of stimulating production of β-endorphin, the method comprising applying the sanshool, the isomer thereof, the hydrate thereof, or the solvate thereof to a subject in need thereof in an effective dose. Another embodiment may provide, as an active ingredient for use in a composition for stimulating production of β-endorphin, the sanshool, the isomer thereof, the hydrate thereof, or the solvate thereof. Also, non-therapeutic use of the sanshool, the isomer thereof, the hydrate thereof, or the solvate thereof as an active ingredient for stimulating production of β-endorphin may be provided.

In an embodiment, the sanshool is an alkamide compound having four or more conjugated double bonds separated from Zanthoxylum piperitum. An embodiment of the present disclosure may comprise, as the sanshool, at least one selected from the group consisting of alpha (α)-sanshool, hydroxy-α-sanshool, dihydroxy-α-sanshool, β-sanshool, hydroxy-β-sanshool, dihydroxy-β-sanshool, gamma (γ)-sanshool, hydroxy-γ-sanshool, hydroxy-γ-isosanshool, dihydroxy-γ-sanshool, delta (5)-sanshool, hydroxy-epsilon (ε)-sanshool, and hydroxy-zeta (ζ)-sanshool. The chemical structure of each sanshool is shown in the following table.

TABLE 1

| | |
|---|---|
| α-sanshool | 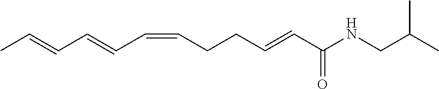 [SMILES: C/C=C/C=C/C=C\CC/C=C/C(=O)NCC(C)C] |
| β-sanshool | 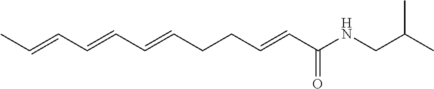 [SMILES: C/C=C/C=C/C=C/CC/C=C/C(=O)NCC(C)C] |
| γ-sanshool | 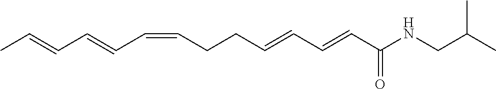 [SMILES: C/C=C/C=C/C=C\CC/C=C/C=C/C(=O)NCC(C)C] |
| δ-sanshool | 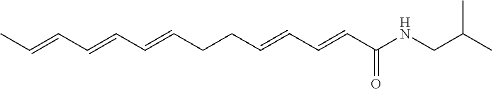 [SMILES: C/C=C/C=C/C=C/CC/C=C/C=C/C(=O)NCC(C)C] |
| hydroxy-α-sanshool | 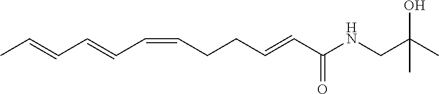 [SMILES: C/C=C/C=C/C=C\CC/C=C/C(=O)NCC(C)(C)O] |
| dihydroxy-α-sanshool | 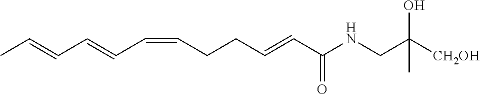 [SMILES: C/C=C/C=C/C=C\CC/C=C/C(=O)NCC(C)(O)CO] |
| hydroxy-β-sanshool | 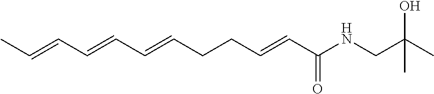 [SMILES: C/C=C/C=C/C=C/CC/C=C/C(=O)NCC(C)(C)O] |
| dihydroxy-β-sanshool | 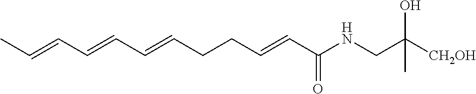 [SMILES: C/C=C/C=C/C=C/CC/C=C/C(=O)NCC(C)(O)CO] |
| hydroxy-γ-sanshool | 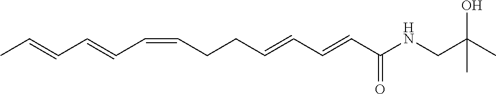 [SMILES: C/C=C/C=C/C=C\CC/C=C/C=C/C(=O)NCC(C)(C)O] |
| hydroxy-γ-isosanshool | 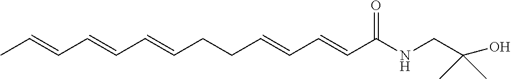 [SMILES: C/C=C/C=C/C=C/CC/C=C/C=C/C(=O)NCC(C)(C)O] |
| dihydroxy-γ-sanshool | 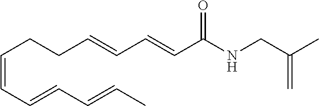 [SMILES: C=C(C)CNC(=O)/C=C/C=C/CC\C=C/C=C/C=C/C] |
| hydroxy-ε-sanshool | 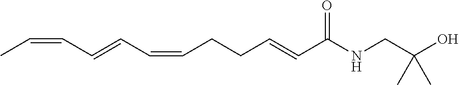 [SMILES: C\C=C/C=C/C=C\CC/C=C/C(=O)NCC(C)(C)O] |
| hydroxy-ζ-sanshool | 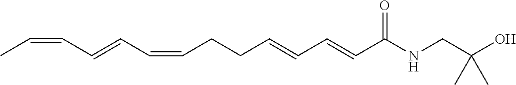 [SMILES: C\C=C/C=C/C=C\CC/C=C/C=C/C(=O)NCC(C)(C)O] |

In an embodiment, content of the sanshool, the isomer thereof, the hydrate thereof, or the solvate thereof is not limited, but may be, for example, in a range of 0.0001 wt % to 1 wt % based on the total weight of the composition. Specifically, the sanshool, the isomer thereof, the hydrate thereof, or the solvate thereof may be comprised in an amount of 0.0001 wt % or more, 0.0002 wt % or more, 0.0003 wt % or more, 0.0004 wt % or more, 0.0005 wt % or more, 0.0006 wt % or more, 0.0007 wt % or more, 0.0008 wt % or more, 0.0009 wt % or more, 0.001 wt % or more, 0.002 wt % or more, 0.003 wt % or more, 0.004 wt % or more, 0.005 wt % or more, 0.006 wt % or more, 0.007 wt % or more, 0.008 wt % or more, 0.009 wt % or more, 0.01 wt % or more, 0.05 wt % or more, 0.1 wt % or more, 0.2 wt % or more, 0.3 wt % or more, 0.4 wt % or more, 0.5 wt % or more, 0.6 wt % or more, 0.7 wt % or more, 0.8 wt % or more, or 0.9 wt % or more, based on the total weight of the composition. Specifically, the sanshool, the isomer thereof, the hydrate thereof, or the solvate thereof may be comprised in an amount of 1 wt % or less, 0.9 wt % or less, 0.8 wt % or less, 0.7 wt % or less, 0.6 wt % or less, 0.5 wt % or less, 0.4 wt % or less, 0.3 wt % or less, 0.2 wt % or less, 0.1 wt % or less, 0.09 wt % or less, 0.08 wt % or less, 0.07 wt % or less, 0.06 wt % or less, 0.05 wt % or less, 0.04 wt % or less, 0.03 wt % or less, 0.02 wt % or less, 0.01 wt % or less, 0.009 wt % or less, 0.008 wt % or less, 0.007 wt % or less, 0.006 wt % or less, 0.005 wt % or less, 0.004 wt % or less, 0.003 wt % or less, 0.002 wt % or less, 0.001 wt % or less, 0.0009 wt % or less, 0.0008 wt % or less, 0.0007 wt % or less, 0.0006 wt % or less, 0.0005 wt % or less, 0.0004 wt % or less, 0.0003 wt % or less, or 0.0002 wt % or less, based on the total weight of the composition. When the sanshool, the isomer thereof, the hydrate thereof, or the solvate thereof are comprised below the range, the effect of stimulating production of β-endorphin may be insignificant, and when the sanshool, the isomer thereof, the hydrate thereof, or the solvate thereof are comprised over the range, skin irritation may occur and formulation stability may be affected.

In an embodiment, a dosage of the sanshool, the isomer thereof, the hydrate thereof, or the solvate thereof is not limited, but may be, for example, in a range of 1 mg/kg/day to 10,000 mg/kg/day when applied to the skin. Specifically, a dosage of the sanshool, the isomer thereof, the hydrate thereof, or the solvate thereof may be 1 mg/kg/day or more, 10 mg/kg/day or more, 20 mg/kg/day or more, 30 mg/kg/day or more, 40 mg/kg/day or more, 50 mg/kg/day or more, 60 mg/kg/day or more, 70 mg/kg/day or more, 80 mg/kg/day or more, 90 mg/kg/day or more, 100 mg/kg/day or more, 200 mg/kg/day or more, 300 mg/kg/day or more, 400 mg/kg/day or more, 500 mg/kg/day or more, 600 mg/kg/day or more, 700 mg/kg/day or more, 800 mg/kg/day or more, 900 mg/kg/day or more, 1,000 mg/kg/day or more, 2,000 mg/kg/day or more, 3,000 mg/kg/day or more, 4,000 mg/kg/day or more, 5,000 mg/kg/day or more, 6,000 mg/kg/day or more, 7,000 mg/kg/day or more, 8,000 mg/kg/day or more, or 9,000 mg/kg/day. Specifically, a dosage of the sanshool, the isomer thereof, the hydrate thereof, or the solvate thereof may be 10,000 mg/kg/day or less, 9,000 mg/kg/day or less, 8,000 mg/kg/day or less, 7,000 mg/kg/day or less, 6,000 mg/kg/day or less, 5,000 mg/kg/day or less, 4,000 mg/kg/day or less, 3,000 mg/kg/day or less, 2,000 mg/kg/day or less, 1,000 mg/kg/day or less, 900 mg/kg/day or less, 800 mg/kg/day or less, 700 mg/kg/day or less, 600 mg/kg/day or less, 500 mg/kg/day or less, 400 mg/kg/day or less, 300 mg/kg/day or less, 200 mg/kg/day or less, 100 mg/kg/day or less, 90 mg/kg/day or less, 80 mg/kg/day or less, 70 mg/kg/day or less, 60 mg/kg/day or less, 50 mg/kg/day or less, 40 mg/kg/day or less, 30 mg/kg/day or less, 20 mg/kg/day or less, or 10 mg/kg/day or less.

The composition according to an embodiment of the present disclosure comprises the sanshool, the isomer thereof, the hydrate thereof, or the solvate thereof as an active ingredient, thereby stimulating production of β-endorphin to soothe the skin from internal and external stress or stimuli factors or relieve skin irritation. Also, the composition according to an embodiment of the present disclosure may stimulate production of β-endorphin, thereby giving a good feeling to the skin and suppressing unpleasant reactions. The composition according to an embodiment of the present disclosure may stimulate production of β-endorphin, thereby stimulating pigmentation of the skin or hair. In the present specification, the expression “stimulating pigmentation” refers to stimulating production of melanin in the skin or hair to make the skin color or hair color darker. The “stimulating pigmentation” may comprise, for example, a skin tanning effect, prevention or improvement of leukoplakia, prevention or improvement of hypochromatism, and stimulating black hair growth, but is not limited thereof.

A composition according to embodiments of the present disclosure may be a composition for external application to the skin comprising the active ingredient. More specifically, the composition may be a composition of a skin application dosage form. In an embodiment, an application area may comprise all tissues covering the body surface of the body, such as the skin, that is, the face, scalp, or body. The sanshool, the isomer thereof, the hydrate thereof, or the solvate thereof, which is an active ingredient of the present disclosure, acts as a cannabinoid receptor type 2 (CB2) agonist present in an epidermal membrane, thereby activating a CB2 receptor to effectively stimulate production of β-endorphin.

A composition according to embodiments of the present disclosure may provide useful effects to the skin desired in the present disclosure even when administered transdermally or orally, in addition to when applied to the skin. In an embodiment, the composition may be a composition for transdermal or oral administration, the composition comprising the active ingredient. Mohab M. Ibrahim et al., PNAS, vol. 102, no. 8, 3093-3098 (2004) has reported that intraperitoneal administration of AM1241 as a CB2 receptor agonist had an effect on inducing endorphin secretion, and that endorphin-induced pain relief was achieved via endorphin receptors on the skin, and Munekage et al., DRUG METABOLISM AND DISPOSITION vol. 39, no. 10, 1784-1788 (2011) has reported that sanshool remained in plasma as a result of confirming movement of a drug in the plasma when a sanshool-containing drug was ingested. The two documents are incorporated herein by reference in their entirety.

In an embodiment, the composition may be a cosmetic composition. In an embodiment, the cosmetic composition according to the present disclosure may be formulated comprising a cosmetically or dermatologically acceptable medium or base. This may be provided in all dosage forms suitable for topical application, for example, in the form of solutions, gels, solids, kneaded anhydrous products, emulsions obtained by dispersing an oily phase in an aqueous phase, suspensions, microemulsions, microcapsules, microgranules, or ionic (liposomes) and non-ionic vesicular dispersants, or in the form of a cream, skin, lotion, powder, ointment, spray, or conceal stick. Also, it may also be used in the form of a foam or in the form of an aerosol composition further comprising a compressed propellant. These compositions may be prepared according to conventional methods in the art.

In an embodiment, the cosmetic composition according to the present disclosure may comprise other ingredients that may preferably give a synergistic effect to the main effect together with the active ingredient within a range that does not impair the main effect, and other ingredients in addition to the active ingredient of the present disclosure may be appropriately selected and mixed by those skilled in the art without difficulty according to the dosage form or purpose of use of other cosmetic compositions. Also, in an embodiment, the cosmetic composition according to the present disclosure may comprise other components commonly mixed in the cosmetic composition, as necessary, in addition to the ingredient. For example, there are moisturizers, emollients, organic and inorganic pigments, organic powders, UV absorbers, preservatives, disinfectants, antioxidants, plant extracts, pH controlling agents, alcohols, pigments, fragrances, blood circulation promoters, cooling agents, antiperspirant, purified water, etc. Other compounding ingredients that may be comprised in the cosmetic composition of the present disclosure are not limited thereto, and a mixing amount of the ingredients may be within a range that does not impair the purpose and effect of the present disclosure.

In another embodiment, the composition may be a pharmaceutical composition. The pharmaceutical composition may further comprise a pharmaceutical adjuvant, such as a preservative, a stabilizer, a wetting agent, or an emulsificant, a salt and/or buffer for regulating osmotic pressure, etc., and other therapeutically useful substances. In an embodiment, the pharmaceutical composition may be an oral administration agent, and the oral administration may comprise, for example, tablets, pills, hard and soft capsules, solutions, suspensions, emulsifiers, syrups, powders, fine granules, granules, and pellets. These dosage forms may comprise, in addition to the active ingredient, a surfactant, a diluent (e.g., lactose, dextrose, sucrose, mannitol, sorbitol, cellulose, and glycine), and a lubricant (e.g., silica, talc, stearic acid and magnesium or calcium salts thereof, and polyethylene glycol). The tablets may also comprise binders such as magnesium aluminum silicate, starch paste, gelatin, tragacanth, methylcellulose, sodium carboxymethylcellulose, and polyvinylpyrrolidine, and in some cases, may comprise pharmaceutical additives such as starch, agar, disintegrants such as alginic acid or sodium salts thereof, absorbents, colorants, flavoring agents, and sweetening agents. The tablets may be prepared by a conventional mixing, granulating, or coating method. In an embodiment, the pharmaceutical composition may be a parenteral administration agent, and the parenteral administration agent may be in the dosage form of an ointment, skin, lotion, gel, cream, spray, suspension, emulsion, patch, and the like, but is not limited thereto. In an embodiment, a dosage of the pharmaceutical composition may vary according to the age, gender, and weight of a subject to be treated, the specific disease or pathological condition to be treated, the severity of the disease or pathological condition, the route of administration, and the determination of a prescriber. Dosage determination based on these factors is within the level of those skilled in the art. For example, the dosage may be from 1 mg/kg/day to 10,000 mg/kg/day, but the dosage does not limit the scope of the present specification in any way.

In another embodiment, the composition may be a food composition. For example, the composition may be processed into functional foods such as fermented milk, cheese, yogurt, juice, probiotics, and health food, and may be used in the form of various other food additives. In an embodiment, the composition may be a composition for health food. In an embodiment, the composition for health food may be formulated as a pill, a capsule, a tablet, a granule, a caramel, or a drink. In another embodiment, the composition for health food may be processed in the form of a solution, a powder, a granule, a tablet, or a tea bag. The composition may be administered by various methods such as simple drinking, injection administration, spray method, or squeeze method. The composition may comprise other ingredients that may give a synergetic effect to the main effect within a range that does not impair the main effect of the present disclosure. For example, in order to improve physical properties, an additive such as a fragrance, a pigment, a disinfectant, an antioxidant, a preservative, a moisturizer, a viscosity increasing agent, a mineral dye, an emulsifier, and a synthetic polymer substance may be further comprised. In addition, an auxiliary component such as a water-soluble vitamin, an oil-soluble vitamin, a high molecular peptide, a high molecular polysaccharide, and a seaweed extract may be further comprised. The ingredients may be selected and mixed by those skilled in the art according to the dosage form or purpose of use, and an addition amount thereof may be selected within a range that does not impair the purpose and effect of the present disclosure. For example, an addition amount of the ingredients may be from 0.0001 wt % to 99.9 wt % based on the total weight of the composition.

Hereinafter, the present disclosure is described in detail with reference to Examples, Comparative Examples, and Experimental Examples. These are only presented by way of example to explain the present disclosure in more detail, and it is obvious to those skilled in the art that the scope of the present disclosure is not limited by these Examples, Comparative Examples, and Experimental Examples.

### Experimental Example 1

In an embodiment of the present disclosure, the following experiment was conducted to confirm the effect of stimulating production of β-endorphin when sanshool is applied to the skin.

First, normal human epidermal cells, keratinocyte (Gibco), were cultured from a 6-well plate to a confluence point at 37° C. under 5% CO₂ culture conditions. In an embodiment of the present disclosure, a keratinocyte-SFM (Gibco) medium was treated with 10 μM of each of hydroxy-α-sanshool (CHEMFACES®; Example 1), hydroxy-γ-sanshool (Chengdu Biopurify Phytochemicals Ltd.; Example 2), hydroxy-β-sanshool (Chengdu Biopurify Phytochemicals Ltd.; Example 3), hydroxy-ε-sanshool (Chengdu Biopurify Phytochemicals Ltd.; Example 4), cannabidiol (CBD) (Sigma-Aldrich®; Comparative Example 1), honokiol (Sigma-Aldrich®; Comparative Example 2), magnolol (Sigma-Aldrich®; Comparative Example 3), and palmitoylethanolamide (PEA) (Sigma-Aldrich®; Comparative Example 4), which are known as CB2 agonists as Comparative Examples, and cultured at 37° C. for six hours under 5% CO₂ culture conditions. Afterwards, each medium was collected, and a β-endorphin EIA kit (EK-022-14, Phoenix pharmaceuticals, Belmont, Calif.) was used to quantify an amount of β-endorphin secreted from keratinocyte in each medium. Student t-test was used for statistical significance between specimens, and when a value of p-value is less than or equal to 0.05, it was analyzed as statistically significant. (*p<0.05, **p<0.01, ***p<0.001)

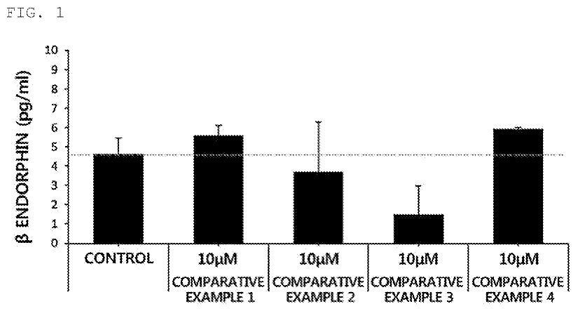

As a result, as in FIG. 1, it may be confirmed that honokiol (Comparative Example 2) and magnolol (Comparative Example 3) among Comparative Examples of the present disclosure, which are known as CB2 agonists, show a further decrease in an amount of β-endorphin secretion compared to a control group, so substances known to activate a CB2 receptor do not necessarily stimulate production of β-endorphin.

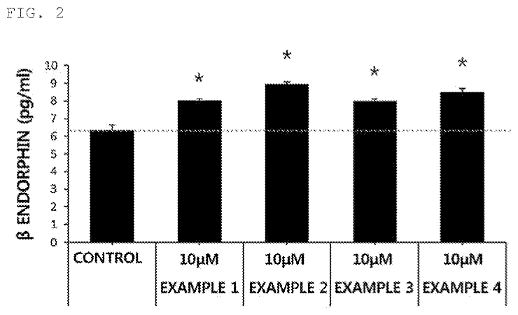

Also, as in FIG. 2, it may be confirmed that sanshool of Examples 1 to 4, which is an embodiment of the present disclosure, has been shown to stimulate production of β-endorphin in epidermal cells, and is extremely higher in efficacy of stimulating production of β-endorphin than CBD (Comparative Example 1) and PEA (Comparative Example 4) of FIG. 1.

### Experimental Example 2

In an embodiment of the present disclosure, the following experiment was conducted to confirm the effect of stimulating production of β-endorphin upon application to the skin according to different concentrations of sanshool.

First, normal human epidermal cells, keratinocyte (Gibco), were cultured from a 6-well plate to a confluence point at 37° C. under 5% CO₂ culture conditions. In an embodiment of the present disclosure, a keratinocyte-SFM medium (Gibco) was treated with each of 0.00006 wt % and 0.0006 wt % of hydroxy-α-sanshool, and cultured at 37° C. for six hours under 5% CO₂ culture conditions. Afterwards, each medium was collected, and a β-endorphin EIA kit (EK-022-14, Phoenix pharmaceuticals, Belmont, Calif.) was used to quantify an amount of β-endorphin secreted from keratinocyte in each medium. Student t-test was used for statistical significance between specimens, and when a value of p-value is less than or equal to 0.05, it was analyzed as statistically significant. (*p<0.05, **p<0.01, ***p<0.001)

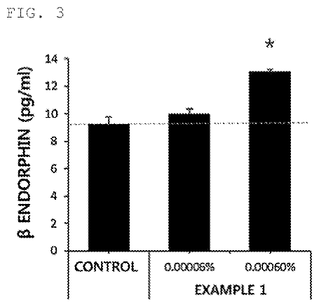

As a result, as in FIG. 3, it was confirmed that when sanshool was comprised in an amount of 0.00006 wt %, β-endorphin production effect was not shown, whereas when sanshool was comprised in an amount of 0.006 wt %, production of β-endorphin was induced.

### Experimental Example 3

In an embodiment of the present disclosure, the following experiment was conducted to confirm skin soothing and irritation relief effects upon application to the skin according to different concentrations of sanshool.

First, a human-derived keratinocyte, HaCaT, was dispensed in a 24-well plate together with a Dulbecco's modified eagle's medium (DMEM) containing 10% fetal bovine serum (FBS), and cultured at 37° C. under 5% CO₂ culture conditions for 24 hours. Afterwards, while inducing allergic contact dermatitis conditions to the HaCat with 15 ng/ml of poly (I:C), each of Example 1 (hydroxy-α-sanshool) and Example 2 (hydroxy-γ-sanshool), which were each dissolved in dimethyl sulfoxide (DMSO), were treated at 10 μM and dissolved in the medium. After 24 hours, each medium was collected, and IL-1b ELISA kit (R&D systems) was used to measure an amount of cytokine IL-1b according to a manufacturer's method.

TABLE 2

| Specimen | | Concentration | IL-1b (pg/ml) |
|---|---|---|---|
| Purified water | DMSO | 0.1% | 11.0060916 |
| Poly (I:C) | DMSO | 0.1% | 11.6454813 |
| 15 ng/ml | Example 1 (Hydroxy- | 10 μM | 11.2919098 |
| | α-sanshool) | | |
| | Example 2 (Hydroxy- | 10 μM | 11.2000237 |
| | γ-sanshool) | | |

As a result, it was confirmed that sanshools according to an embodiment of the present disclosure showed the effect of reducing excessive cytokine IL-1b induced by poly(I:C), and had effects of soothing the skin and relieving irritation.

Formulation examples of the composition according to an embodiment of the present specification is described below, but other various dosage forms are also applicable, which is not intended to limit the present specification, but is merely intended to describe the present specification in detail.

### [Formulation Example 1] Emulsifying Emollient (Skin Lotion)

An emulsifying emollient was prepared in a conventional method according to the composition described in the following table.

TABLE 3

| Compounding ingredient | Content (wt %) |
|---|---|
| Hydroxy-γ-sanshool | 0.1 |
| Glycerin | 3.0 |
| Butylene glycol | 2.0 |
| Propylene glycol | 2.0 |
| Carboxyvinyl polymer | 0.1 |
| PEG-12 nonyl phenyl ether | 0.2 |
| Polysorbate 80 | 0.4 |
| Ethanol | 10.0 |
| Triethanolamine | 0.1 |
| Preservative, Pigment, Fragrance | Suitable amount |
| Purified water | Balance |

### [Formulation Example 2] Nourishing Emollient (Milk Lotion)

A nourishing emollient was prepared in a conventional method according to the composition described in the following table.

TABLE 4

| Compounding ingredient | Content (wt %) |
|---|---|
| Hydroxy-α-sanshool | 0.1 |
| Glycerin | 3.0 |
| Butylene glycol | 3.0 |
| Propylene glycol | 3.0 |
| Carboxyvinyl polymer | 0.1 |
| Beeswax | 4.0 |
| Polysorbate 60 | 1.5 |
| Caprylic/capric triglyceride | 5.0 |
| Squalane | 5.0 |
| Sorbitan sesquioleate | 1.5 |
| Liquid paraffin | 0.5 |
| Cetearyl alcohol | 1.0 |
| Triethanolamine | 0.2 |
| Preservative, Pigment, Fragrance | Suitable amount |
| Purified water | Balance |

### [Formulation Example 3] Massage Cream

A massage cream was prepared in a conventional method according to the composition described in the following table.

TABLE 5

| Compounding ingredient | Content (wt %) |
|---|---|
| Hydroxy-γ-sanshool | 0.1 |
| Glycerin | 8.0 |
| Butylene glycol | 4.0 |
| Liquid paraffin | 45.0 |
| β-glucan | 7.0 |
| Carbomer | 0.1 |
| Caprylic/capric triglyceride | 3.0 |
| Beeswax | 4.0 |
| Cetearyl glucoside | 1.5 |
| Sorbitan sesquioleate | 0.9 |
| Vaseline | 3.0 |
| Paraffin | 1.5 |
| Preservative, Pigment, Fragrance | Suitable amount |
| Purified water | Balance |

### [Formulation Example 4] Ointment

An ointment was prepared in a conventional method according to the composition described in the following Table 6.

TABLE 6

| Raw material name | Content (wt %) |
|---|---|
| Glycerin | 8.0 |
| Butylene glycol | 4.0 |
| Liquid paraffin | 15.0 |
| β-glucan | 7.0 |
| Carbomer | 0.1 |
| Hydroxy-γ-sanshool | 0.1 |
| Caprylic/capric triglyceride | 3.0 |
| Squalane | 1.0 |
| Cetearyl glucoside | 1.5 |
| Sorbitan stearate | 0.4 |
| Cetearyl alcohol | 1.0 |
| Preservative | Suitable amount |
| Fragrance | Suitable amount |
| Pigment | Suitable amount |
| Beeswax | 4.0 |
| Purified water | Balance |
| Total | 100 |

### [Formulation Example 5] Tablet

100 mg of hydroxy-γ-sanshool, 400 mg of lactose, 400 mg of corn starch, and 2 mg of magnesium stearate were mixed, and then tableted according to a conventional tablet preparing method, thereby preparing a tablet.

### [Formulation Example 6] Capsule

100 mg of hydroxy-α-sanshool, 400 mg of lactose, 400 mg of corn starch, and 2 mg of magnesium stearate were mixed, and then filled into a gelatin capsule according to a conventional capsule preparation method, to thereby prepare a capsule.

### [Formulation Example 7] Granule

50 mg of hydroxy-γ-sanshool, 250 mg of anhydrous crystalline glucose, and 550 mg of starch were mixed, formed into a granule by using a fluidized bed granulator, and then filled in a bag.

### [Formulation Example 8] Drink

50 mg of hydroxy-α-sanshool, 10 g of glucose, 0.6 g of citric acid, and 25 g of liquid oligosaccharide were mixed, 300 ml of purified water was added thereto, and then the mixture was filled in each bottle by 200 ml. After filling the bottle, the mixture was sterilized at 130° C. for 4 seconds to 5 seconds to thereby prepare a drink.

### [Formulation Example 9] Caramel

50 mg of hydroxy-γ-sanshool, 1.8 g of corn syrup, 0.5 g of skim milk, 0.5 g of soybean lecithin, 0.6 g of butter, 0.4 g of hydrogenated vegetable oil, 1.4 g of sugar, 0.58 g of margarine, and 20 mg of salt were mixed and then caramelized.

### [Formulation Example 10] Health Food

TABLE 7

| Ingredient | Content | |
|---|---|---|
| Hydroxy-γ-sanshool | 100 | mg |

Vitamin mixture

| | | |
|---|---|---|
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folate | 50 | μg |
| Calcium pantothenate | 0.5 | mg |

Mineral mixture

| | | |
|---|---|---|
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium Citrate | 90 | mg |
| Calcium carbonate | 100 | mg |
| Magnesium chloride | 24.8 | mg |

Although a composition ratio of the vitamin and mineral mixture was set by mixing ingredients relatively suitable for health food as an example, a mixing ratio thereof may be arbitrarily modified, and after mixing the ingredients according to a conventional health food preparation method, may be used for preparing a granule and preparing a health food composition according to a conventional method.

### [Formulation Example 11] Health Drink

TABLE 8

| Ingredient | Content | |
|---|---|---|
| Hydroxy-α-sanshool | 10 | mg |
| Citric acid | 1,000 | mg |
| Oligosaccharides | 100 | g |
| Plum concentrate | 2 | g |
| Taurine | 1 | g |
| Purified water | Balance | |
| Total volume | 900 | ml |

As shown in the table, the remaining amount of purified water is added to make a total volume of 900 ml and the ingredients are mixed according to a conventional health drink preparation method, followed by stirring and heating at 85° C. for an hour, and then a resulting solution is filtered, obtained in a sterilized 2 L container, sealed and sterilized, and then stored in a refrigerator for use in the manufacture of health drink composition.

### [Formulation Example 12] Injection

An injection was prepared in a conventional method according to the composition described in the following table.

TABLE 9

| Compounding ingredient | Content |
|---|---|
| Hydroxy-γ-sanshool | 10-50 mg |
| Sterilized distilled water for injection | Suitable amount |
| pH controlling agent | Suitable amount |

The present disclosure may provide the following embodiments as an embodiment.

A first embodiment provides a method for stimulating production of β-endorphin, the method comprising applying sanshool, an isomer thereof, a hydrate thereof, or a solvate thereof to a subject in need thereof in an effective amount.

A second embodiment provides the method of the first embodiment, wherein the sanshool is at least one selected from the group consisting of α-sanshool, hydroxy-α-sanshool, dihydroxy-α-sanshool, β-sanshool, hydroxy-β-sanshool, dihydroxy-β-sanshool, γ-sanshool, hydroxy-γ-sanshool, hydroxy-γ-isosanshool, dihydroxy-γ-sanshool, δ-sanshool, hydroxy-ε-sanshool, and hydroxy-ζ-sanshool.

A third embodiment provides the method of the first embodiment or the second embodiment, wherein, in the method, the sanshool, the isomer thereof, the hydrate thereof, or the solvate thereof are comprised as an active ingredient in a composition, and the active ingredient is comprised in an amount of 0.0001 wt % to 1 wt % based on the total weight of the composition.

A fourth embodiment provides the method of any one of the first embodiment to the third embodiment, wherein a dosage of the sanshool, the isomer thereof, the hydrate thereof, or the solvate thereof is from 1 mg/kg/day to 10,000 mg/kg/day.

A fifth embodiment provides the method of any one of the first embodiment to the fourth embodiment, wherein the method is for soothing skin or relieving skin irritation by stimulating the production of the β-endorphin.

A sixth embodiment provides the method of any one of the first embodiment to the fifth embodiment, wherein the method is for stimulating pigmentation of skin or hair by stimulating the production of the β-endorphin.

A seventh embodiment provides the method of any one of the first embodiment to the sixth embodiment, wherein the method comprises applying the sanshool, the isomer thereof, the hydrate thereof, or the solvate thereof to skin of a subject.

An eighth embodiment provides the method of any one of the first embodiment to the seventh embodiment, wherein the method comprises applying the sanshool, the isomer thereof, the hydrate thereof, or the solvate thereof by transdermal or oral administration to a subject.

A ninth embodiment provides the method of any one of the first embodiment to the eighth embodiment, wherein the sanshool, the isomer thereof, the hydrate thereof, or the solvate thereof is comprised as an active ingredient in a composition, and the composition is a cosmetic composition.

A tenth embodiment provides the method of any one of the first embodiment to the ninth embodiment, wherein the sanshool, the isomer thereof, the hydrate thereof, or the solvate thereof is comprised as an active ingredient in a composition, and the composition is a pharmaceutical composition.

An eleventh embodiment provides the method of any one of the first embodiment to the tenth embodiment, wherein the sanshool, the isomer thereof, the hydrate thereof, or the solvate thereof is comprised as an active ingredient in a composition, and the composition is a food composition.

What is claimed is:

1. A method for stimulating production of beta (β)-endorphin, the method comprising applying an effective dose of sanshool, an isomer thereof, a hydrate thereof, or a solvate thereof to a subject in need thereof.

2. The method according to claim 1, wherein the sanshool is at least one selected from the group consisting of alpha (α)-sanshool, hydroxy-α-sanshool, dihydroxy-α-sanshool, β-sanshool, hydroxy-β-sanshool, dihydroxy-β-sanshool, γ-sanshool, hydroxy-γ-sanshool, hydroxy-γ-isosanshool, dihydroxy-γ-sanshool, delta (δ)-sanshool, hydroxy-epsilon (ε)-sanshool, and hydroxy-zeta (ζ)-sanshool.

3. The method according to claim 1, wherein the sanshool, the isomer thereof, the hydrate thereof, or the solvate thereof is comprised as an active ingredient in a composition, and
the active ingredient is comprised in an amount of 0.0001 wt % to 1 wt % based on a total weight of the composition.

4. The method according to claim 1, wherein a dosage of the sanshool, the isomer thereof, the hydrate thereof, or the solvate thereof is from 1 mg/kg/day to 10,000 mg/kg/day.

5. The method according to claim 1, wherein the method is for soothing skin or relieving skin irritation by stimulating the production of the β-endorphin.

6. The method according to claim 1, wherein the method is for stimulating pigmentation of skin or hair by stimulating the production of the β-endorphin.

7. The method according to claim 1, wherein the method comprises applying the sanshool, the isomer thereof, the hydrate thereof, or the solvate thereof to skin of a subject.

8. The method according to claim 1, wherein the method comprises applying the sanshool, the isomer thereof, the hydrate thereof, or the solvate thereof by transdermal or oral administration to a subject.

9. The method according to claim 1, wherein the sanshool, the isomer thereof, the hydrate thereof, or the solvate thereof is comprised as an active ingredient in a composition, and
the composition is a cosmetic composition.

10. The method according to claim 1, wherein the sanshool, the isomer thereof, the hydrate thereof, or the solvate thereof is comprised as an active ingredient in a composition, and
the composition is a pharmaceutical composition.

11. The method according to claim 1, wherein the sanshool, the isomer thereof, the hydrate thereof, or the solvate thereof is comprised as an active ingredient in a composition, and
the composition is a food composition.
